(19) United States
(12) Patent Application Publication
(10) Pub. No.: US 2014/0303260 A1
(43) Pub. Date: Oct. 9, 2014

(54) PHARMACEUTICAL COMPOSITIONS OF RESVERATROL

(71) Applicant: NAD Life Pty Ltd, Thornleigh New Wouth Wales (AU)

(72) Inventors: Ross Stewart Grant, Kellyville New South Wales (UA); Keith Lindbeck, Cooranbong New South Wales (AU)

(21) Appl. No.: 14/352,822

(22) PCT Filed: Oct. 17, 2012
(86) PCT No.: PCT/AU2012/001253

§ 371 Date: Apr. 18, 2014

(30) Foreign Application Priority Data
Oct. 19, 2011 (AU) 2011904326

(51) Int. Cl. A61K 9/00; A61K 9/68; A23L 2/385; A61K 31/05

(52) U.S. Cl. CPC A61K 9/0056; A61K 31/05; A61K 9/0058; A23L 2/385
USPC 514/733; 568/729

(57) ABSTRACT

A pharmaceutical composition for administering a therapeutically effective amount of resveratrol or a functionally equivalent analogue or derivative thereof to a subject. The absorption of resveratrol occurs through the subject's buccal/sublingual membranes, thereby by-passing first pass metabolism by the liver.

### TECHNICAL FIELD

The invention relates to compositions of resveratrol that are adapted to be administered to a person. Specifically, the present invention relates to compositions of resveratrol that avoid first pass metabolism to promote maximal systemic circulation of resveratrol and promotion of NAD synthesis providing benefit to NAD associated cellular biochemistry including, redox couples (e.g. NAD/NADH), ADP ribosylation reactions (e.g. PARP, CD38 etc), deacetylase activity (e.g. sirtuins) and NAD⁺ facilitated neurotransmission.

The invention further relates to methods and pharmaceutical compositions for the prevention and treatment of conditions and diseases associated with either reduced NAD⁺ synthesis (as may occur in aging tissue) or increased NAD+ turnover (e.g. oxidative stress and/or DNA damage significantly increases NAD+ catabolism) by administration of resveratrol.

### BACKGROUND ART

It is known that NAD plays an important role in over five hundred biochemical processes including energy production, repair of broken DNA activation of the sirtuin longevity enzymes, immune cell signaling (through CD38) and more recently as a neurotransmitter. Importantly, the Applicant has discovered that NAD⁺ levels fall considerably with older age.

For example, see “Age-Associated Changes In Oxidative Stress and NAD(+) Metabolism In Human Tissue” Massudi H, Grant R, Braidy N, Guest 3, Farnsworth B, Guillemin G J. PLoS One. 2012; 7 (7):e42357. Epub 2012Jul. 27 and “Age related changes in NAD+ metabolism oxidative stress and Sirt1 activity in wistar rats” Braidy N, Guillemin G J, Mansour H, Chan-Ling T, Poljak A, Grant R. PLoS One. 2011 Apr. 26; 6 (4):e19194.

Accordingly, it is highly desirable for the body to more efficiently manufacture NAD, particularly as both sirtuin activity and PARP activity use NAD+ as the substrate for their enzyme activity.

The Applicant's previous International PCT patent application (publication no. WO 2009/108999) describes a method of inducing NAD synthesis in a subject by upregulation of nicotinamide mononucleotide adenylyl transferase (NMNAT) activity by administering to the subject a therapeutically effective amount of resveratrol or a functionally equivalent analogue or derivative thereof.

This prior application provides a detailed discussion of the mechanism of action of resveratrol in inducing NAD+ synthesis and so is incorporated herein by reference.

In view of the importance of resveratrol in increasing NAD synthesis in the body, it is desirable to provide an efficient and effective mechanism of administering resveratrol to a person.

### DISCLOSURE OF INVENTION

According to the present invention there is provided a pharmaceutical composition for administering a therapeutically effective amount of resveratrol or a functionally equivalent analogue or derivative thereof to a subject characterised in that the absorption of resveratrol occurs through the subject's buccal/sublingual membranes, thereby by-passing first pass metabolism by the liver.

In one embodiment, the pharmaceutical composition of the present invention the resveratrol is provided in a gel based confectionery, which is adapted to be at least partially dissolved in the subject's mouth cavity such that the resveratrol is absorbed by the buccal/sublingual membranes.

Preferably, the pharmaceutical composition provides a minimum dosage administration time of about 5 min to allow the resveratrol to be effectively absorbed across the buccal/sublingual membranes. Most preferably, the pharmaceutical composition is adapted to remain in the subject's mouth cavity for about 5 min before completely dissolving.

Preferably, the pharmaceutical composition comprises corn syrup, sugar, fruit juice concentrate, citric acid, flavours, colours, carnauba wax, resveratrol and one or more of gelatine, carrageenan, xanthan, pectin or gellan gum. Most preferably, the one or more of gelatine, carrageenan, xanthan, pectin or gellan gum is present in a concentration sufficient that the pharmaceutical composition maintains its structure when the mixture of ingredients is dried.

Preferably, the resveratrol is in an amount from 0.4% to 5% of finished product weight, thereby providing a resveratrol dosage range of between 20-250 mg/5g level.

Preferably, the resveratrol is mixed with the fruit juice concentrate to form a slurry prior to being added to the mixture, as resveratrol is almost insoluble in aqueous solutions.

According to a second embodiment of the present invention, the resveratrol is provided in a chewing gum, which is adapted to be chewed by the subject so that the resveratrol is absorbed by the buccal/sublingual membranes.

According to the second embodiment, the pharmaceutical composition comprises a gum base (15-25%), a sugar or sugar alternative (50-70%), corn or glucose syrup (15-25%), flavouring (1-5%) and resveratrol (0.6-2.0%).

Most preferably, the pharmaceutical composition comprises a gum base (about 20%), a 30:1 (sugar/resveratrol) mix (about 62%), corn syrup (about 15%) and flavouring (about 1%).

Preferably, the ingredients are mixed together at a temperature of between 75° C. to 80° C., and most preferably at a temperature about 80° C.

Preferably, the subject's NAD level increases by between 30% and 50% within about 60 min of administration of the composition, and most preferably the subject's NAD level increases by between 35% and 45% within about 60 min of administration of the composition.

The subject's NAD level preferably increases by 41% within about 60 min of administration of the composition, and preferably the subject's raised NAD level remains between 30% and 40% above the baseline NAD level for between 1 and 4 hours and decreases at a rate of 1 ng/mL/hr to 3 ng/mL/hr. Most preferably, the subject's raised NAD level remains 35% above the baseline NAD level for 4hrs and decreases at a rate of 2 ng/mL/hr.

A further aspect of the present invention is a method for the treatment of a disease or condition associated with either reduced NAD+ synthesis and/or increased NAD catabolism (usage) or increased requirement for NAD+, such as DNA damage by upregulation of nicotinamide mononucleotide adenylyl transferase (NMNAT) activity by administering to a subject a pharmaceutical composition according to the present invention.

Although the invention is described above with reference to specific embodiments, it will be appreciated by those skilled in the art that it is not limited to those embodiments, but may be embodied in many other forms.

### DEFINITIONS

As used in this application, the singular form “a”, “an” and “the” include plural references unless the context clearly dictates otherwise. For example, the term “a stem cell” also includes a plurality of stem cells.

As used herein, the term “comprising” means “including.” Variations of the word “comprising”, such as “comprise” and “comprises,” have correspondingly varied meanings. Thus, for example, a polynucleotide “comprising” a sequence encoding a protein may consist exclusively of that sequence or may include one or more additional sequences.

As used herein, the term “resveratrol” encompasses either the cis-isomer of resveratrol, the trans-isomer of resveratrol, or a mixture of the two isomers. The term encompasses both the naturally occurring and chemically synthesized active agent and the compound as it may be in the laboratory. Further, when the term “resveratrol” is used herein, it is intended to encompass pharmacologically acceptable salts, esters, amides, prodrugs and derivatives and analogues of resveratrol.

As used herein, the term “synergistic” refers to a greater than additive effect that is produced by a combination of the agents, which exceeds the effect that would otherwise result from use of the agents alone.

A “therapeutically effective amount”, as used herein, includes within its meaning a non-toxic but sufficient amount of the particular therapeutic compound to which it is referring to provide the desired therapeutic effect. The exact amount required will vary from subject to subject depending on factors such as the patient's general health, the patient's age and the stage and severity of the condition.

As used herein, the term “neurodegenerative disorder” refers to a disease or condition in an animal wherein there is a degeneration or inactivation of nerve cells in any location of the body including the brain, central nervous system and periphery.

As used herein, the term “oxidative stress” is used in general context and refers to enhanced generation of free radicals or reactive oxygen species (ROS) (such as α-hydroxy ethyl radical, hydrogen peroxide, peroxy radical, hydroxy radical, and superoxide radical) and/or a depletion in antioxidant defense system causing an imbalance between pro-oxidants and antioxidants. In general, oxidative stress involves the accumulation of free-radicals within the cell or the cell environment which may result in oxidative damage. Oxidative stress may arise from biotic (living) and abiotic (non-living) sources, for example, exposure to U.V. or ionising radiation or chemical agents, infection by different infectious agents, inflammation or reduced mitochondrial efficiency

### BRIEF DESCRIPTION OF THE DRAWINGS

A preferred embodiment of the present invention will now be described, by way of an example only, with reference to the accompanying drawings wherein:

FIG. 1 is a graphical representation of the rise in NAD levels in a number of subjects (series 1-7) following administration of a therapeutically effective amount of resveratrol to each of the subjects by way of a pharmaceutical composition of the present invention.

FIG. 2 is a graphical representation of the effect to a number of subjects (series 1-7) NAD levels (the same group of subjects of the graph shown in FIG. 1) following oral administration of resveratrol.

FIG. 3a is a graphical representation of the difference in serum NAD levels (mean±SEM) after administration of resveratrol via the buccal/sublingual route. *** denotes p<0.001, ** denotes p<0.01, as compared to baseline (t=0).

FIG. 3b is a graphical representation of the difference in serum NAD levels (mean±SEM) after administration of resveratrol via the oral route.

FIG. 3c is a graphical comparison of the difference in a subject's NAD levels following oral administration of resveratrol compared with administration through the buccal/sublingual membranes (i.e. a comparison between FIGS. 3a and 3b).

### MODE(S) FOR CARRYING OUT THE INVENTION

The invention relates to the finding that administration of the polyphenol resveratrol to a subject via a route that avoids first pass metabolism is surprisingly more effective in increasing a subject's NAD levels. In particular, the present invention relates to the surprising finding that administration of resveratrol via absorption through the buccal/sublingual membranes is particularly effective in increasing plasma NAD levels within 60 minutes of administration.

It is well known to orally administer resveratrol to a subject, which is then absorbed into the body via the gastrointestinal tract. Conventional administrative forms of resveratrol include capsules, tablets, liquids and oral slurries. However, all of these conventional administrative forms result in the resveratrol being absorbed by the gastrointestinal tract, which is then subject to first pass metabolism by the liver.

Delivery of resveratrol by any route that avoids first pass metabolism will promote maximal exposure of the body's blood and tissues to resveratrol and therefore, our results show, NAD production, which will therefore benefit cellular biochemistry. However, it should be appreciated that some routes may be more efficient overall. For example, intravenous administration is likely to be the most efficient and effective way of administering resveratrol to a subject. Other forms of administration that are effective in by-passing first pass metabolism by the liver include buccal/sublingual administration, intramuscular administration, subcutaneous administration, intrathecal, pulmonary, intranasal (particularly for access to the brain), per rectal and transdermal administration.

Whilst intravenous administration may be the most efficient route of administering resveratrol to a person, there are certain disadvantages in this administrative route. In particular, many people have an aversion to intravenous injections and would prefer an alternative, less intrusive dosage form. Furthermore, intravenous injection will generally require a qualified medical technician to administer the dosage form of resveratrol to the person. Accordingly, intravenous injection is not particularly conducive for administration of resveratrol outside of a medical facility.

Further, whilst intranasal administration of resveratrol has the capacity to increase NAD+ in the central nervous system (CNS) through absorption via the olfactory neurons, thereby traversing the cribriform plate into the CNS, there is also a strong likelihood of resveratrol being absorbed into the systemic circulation via absorption into the micro vasculature of the nasal cavity. There are also limitations on the overall dosage time when administering resveratrol to a subject intranasally. It is difficult to provide a sustained nasal spray, which allows for a sufficient dosage time to administer therapeutically optimal amounts of resveratrol to a subject.

Therefore, it is desirable to provide a conveniently administrable dosage form of resveratrol that is effective in delivering resveratrol to a subject and avoids first pass metabolism by the liver.

One such particularly effective pharmaceutical composition is a dosage form that is administered to a person through the buccal/sublingual membranes.

According to a first embodiment of the invention, this pharmaceutical composition is preferably a gel based “gummy” confectionery, which is adapted to be at least partially dissolved relatively slowly in a person's mouth in order to allow the resveratrol sufficient time to be absorbed through the buccal/sublingual membranes.

In a second embodiment, the pharmaceutical composition of the present invention is a chewing gum, which is to be chewed over a sufficient course of time to allow the resveratrol to be absorbed through the person's buccal/sublingual membranes.

Whilst not preferred, other embodiments of the present invention include other suitable dosage forms of resveratrol which are administered through the buccal/sublingual membranes. For example, oral strips which adhere to the roof of the oral cavity, oral/nasal sprays, lozenges, chews and other similar dosage means that remain in a person's mouth cavity for suitable time to allow for a therapeutically sufficient level of resveratrol to be absorbed through the buccal/sublingual membranes.

Both preferred embodiments, and indeed all other possible embodiments, of the pharmaceutical composition of the present invention are designed to allow the resveratrol to be absorbed through the person's buccal/sublingual membranes. This is achieved by the pharmaceutical composition being retained in the person's mouth cavity (i.e. being at least partially dissolved or being chewed) for a sufficient time to allow the resveratrol to be absorbed through the buccal/sublingual membranes.

The minimum time that is required for pharmaceutical composition to remain in a person's mouth cavity to allow the resveratrol to be effectively absorbed across the buccal/sublingual membranes is about 5 min.

According to a preferred embodiment of the first aspect of the present invention, the gel based “gummy” pharmaceutical composition includes corn syrup, sugar, fruit juice concentrate (preferably apple, but other fruit juice concentrates can also be utilised), gelatin (or similar gelling agents), resveratrol, citric acid, flavours, colours, carnauba wax. The relative percentages of each of the major ingredients may be varied to achieve variation in consistency, sweetness, appearance. Gelatine or an equivalent gum or gelling agent (such as carrageenan, xanthan, pectin or gellan gum) must be present in a concentration sufficient to maintain structure when the mixture is dried. Resveratrol concentration may be varied within the confection from levels as low as 20 mg/5 g portion (0.4%) up to levels as high as 250 mg/5 g portion (5%), thus allowing simple variation in dosage levels of the pharmaceutical composition of the present invention. The ingredients are mixed together at a temperature of between 75° C. to 80° C., and preferably at a temperature of about 80° C.

### Example Formulation

A typical formulation of a gel based confectionery made in a small scale batch of about 505 g according to the present invention includes cane sugar in an amount of 180 g (approximately 36% w/w); apple juice (concentrate) in an amount of 160 ml (approximately 32% w/w); corn Syrup in an amount of 100 g approximately 20% w/w); gelatine in an amount of 40g (approximately 8% w/w); resveratrol in an amount of 15 g approximately 3% w/w); citric acid in an amount of 10 g approximately 2% w/w); and flavouring and colouring additives as required to reach desired flavour and appearance.

There are several ways in which resveratrol may be added to the “gummy” mix. However, since resveratrol is almost insoluble in aqueous solutions one particularly preferred method is to mix it as slurry. This slurry may be formed by a combination of fruit juice and resveratrol. Once combined, this slurry is then added to the hot gel product just before depositing into starch moulds.

According to a preferred embodiment of the second aspect of the present invention, the pharmaceutical composition is a chewing gum. Whilst the traditional/natural source of “gum” in chewing gum is chicle, which is obtained from the sap of the sapodilla tree found in Mexico and Guatemala, in more recent times, the modern “gum” in chewing gum is synthesised from styrene-butadiene rubber, which has an equivalent temperature profile to chicle.

According to a preferred embodiment of the second aspect of the present invention, the pharmaceutical composition is a chewing gum that typically includes a gum base (from 15-25%), a sweetness source, which may be sugar, sugar alternatives such as sucralose or erythritol, or a combination of these (from 50-70%); a syrup, such as corn or glucose syrup (15-25%), flavouring such as mint, peppermint, strawberry and similar (at a level to satisfy taste, which is typically 1-5%) and resveratrol (at levels from 0.6-2.0% to allow for variation in dosage levels). The ingredients are mixed together at a temperature of between 75° C. to 80° C., and preferably at a temperature of about 80° C.

### Example Formulation

A typical formulation of a chewing gum made in a small scale batch of about 155 g according to the present invention includes a gum base in an amount of 25 g (approximately 16% w/w); glucose syrup in an amount of 25 g (approximately 16% w/w); icing sugar in an amount of 100 g (approximately 65% w/w); resveratrol in an amount of 3 g (approximately 2% w/w); and flavouring in an amount of 2 g (approximately 1% w/w).

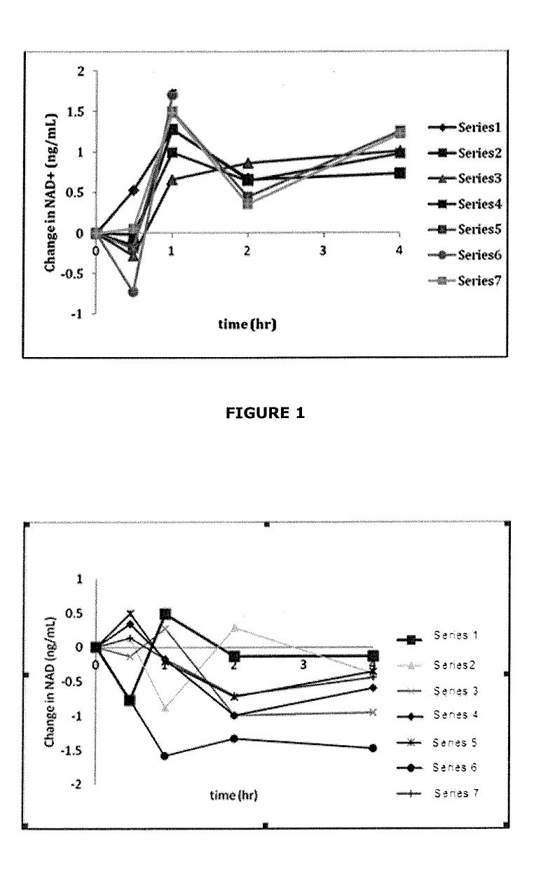

FIG. 1 shows the effect on a test group of subject's NAD levels when administered resveratrol in a pharmaceutical composition according to the first embodiment of the present invention.

It can clearly be seen that the administration of the pharmaceutical composition of the present invention (where resveratrol is absorbed through the buccal/sublingual membranes), causes a rapid onset rise in NAD levels, as well as a sustained increase in the level of NAD.

Administration of resveratrol resulted in a 41% increase in blood NAD levels at 1 hr (60 min). A 35% increased blood NAD level was sustained at 4 hrs compared to baseline. No observable change in blood NAD levels were observed when resveratrol was delivered via powdered slurry (i.e. gastrointestinal absorption alone).

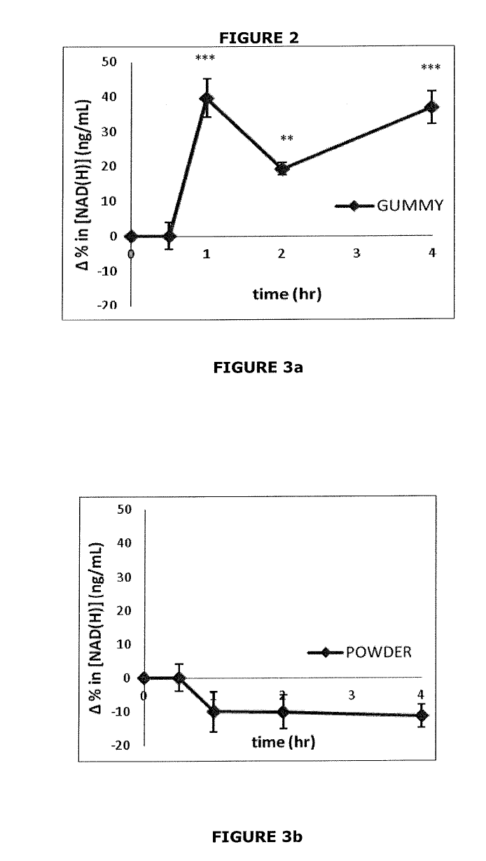

On the other hand, FIG. 2 shows the result of the oral administration of resveratrol to the NAD levels of the same test group of subjects that were administered the pharmaceutical composition of the present invention of FIG. 1.

It can be seen that the oral administration of resveratrol (where it is absorbed through the gastrointestinal tract), provides only a mild increase in the level of NAD in only some of the people in the group. For those people where there is an increase in NAD levels, the increased level relatively quickly decreases back to the baseline level. In some other subjects in the test group, the oral administration of resveratrol had effectively no impact on their NAD levels.

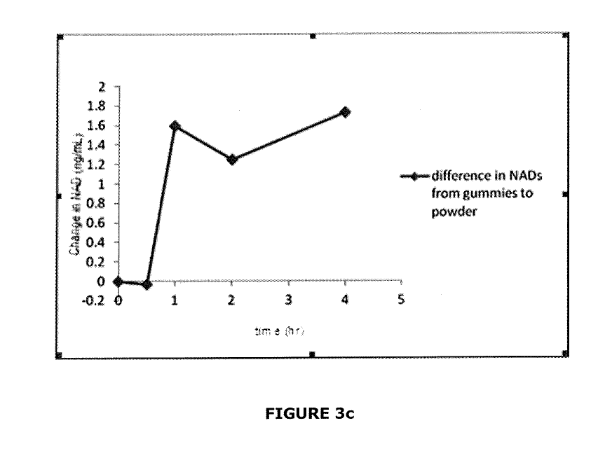

The comparison of the effect on NAD levels between oral administration of resveratrol and buccal/sublingual membrane administration of resveratrol is best shown in FIG. 3. This graph depicts the difference in the change in a subject's NAD levels (in ng/mL) when resveratrol is administered orally (absorption through the gastrointestinal tract) compared to when resveratrol is administrated by the pharmaceutical composition of the present invention (absorption through the buccal/sublingual membranes).

It can be seen from FIG. 3 that the subject's raised NAD level remains between 30% and 40% above the baseline NAD level for between 1 and 4 hours and decreases at a rate of 1 ng/mL/hr to 3 ng/mL/hr following administration of the pharmaceutical composition of the present invention. Most accurately, following administration of the pharmaceutical composition of the present invention, the subject's raised NAD level remains 35% above the baseline NAD level for 4 hrs and decreases at a rate of 2 ng/mL/hr.

It is clear that where the resveratrol is absorbed through the buccal/sublingual membranes, there is a rapid onset rise in NAD levels, as well as a sustained increase in the level of NAD when compared with absorption through the gastrointestinal tract.

### INDUSTRIAL APPLICABILITY

The present invention can be utilised in respect of pharmaceutical compositions for administering resveratrol to induce NAD⁺ synthesis in a subject which may impact essential biochemical processes such as, increased activity of the dehydrogenase enzymes (e.g. alcohol dehydrogenase, lactate dehydrogenase etc), nuclear DNA repair, activation of sirtuins (longevity enzymes) and potentially increased neuronal firing in those neurons where NAD+ serves as a novel neurotransmitter.

Particularly, the invention can be utilised with respect to pharmaceutical compositions for the prevention and treatment of conditions and conditions/diseases associated with either a reduced synthesis of NAD+ (e.g. with age) and/or increased NAD+ usage such as inflammation/oxidative stress and/or DNA damage (also increasing with age) by the administration of resveratrol in a dosage form that by-passes first pass metabolism by the liver.

1. A method for inducing NAD+ synthesis in a subject, said method comprising:
administering to the subject a pharmaceutical composition comprising a therapeutically effective amount of resveratrol or a functionally equivalent analogue or derivative thereof; and
retaining the pharmaceutical composition in the subject's mouth so that absorption of the resveratrol occurs through the subject's buccal/sublingual membranes, thereby by-passing first pass metabolism by the liver.

2. A method of claim 1 wherein the pharmaceutical composition is provided as a gel based confectionary, which is adapted to be at least partially dissolved in the subject's mouth cavity such that the resveratrol is absorbed through the subject's buccal/sublingual membranes.

3. A method of claim 1 or claim 2 wherein the pharmaceutical composition is retained in the subject's mouth for a minimum time of about 5 min.

4. A method of claim 3 wherein the pharmaceutical composition is retained in the subject's mouth for about 5 min before completely dissolving.

5. A method of claim 2 wherein the pharmaceutical composition comprises corn syrup, sugar, fruit juice concentrate, citric acid, flavours, colours, carnauba wax, resveratrol and one or more of gelatine, carrageenan, xanthan, pectin or gellan gum.

6. A method of claim 5 wherein the one or more of gelatine, carrageenan, xanthan, pectin or gellan gum is present in a concentration sufficient that the pharmaceutical composition maintains its structure when the mixture of ingredients is dried.

7. A method of claim 5 wherein the resveratrol is mixed with the fruit juice concentrate to form a slurry prior to being added to the mixture.

8. A method of claim 1 where the resveratrol is in an amount from 0.4% to 5% of finished product weight, thereby providing a resveratrol dosage range of between 20-250 mg/5 g level.

9. A method of claim 1 wherein the pharmaceutical composition is provided as a chewing gum, which is adapted to be chewed by the subject such that the resveratrol is absorbed through the subject's buccal/sublingual membranes.

10. A method of claim 9 wherein the pharmaceutical composition comprises a gum base (15-25%), a sugar or sugar alternative (50-70%), corn or glucose syrup (15-25%), flavouring (1-5%) and resveratrol (0.6-2.0%).

11. A method of claim 10 wherein the pharmaceutical composition comprises a gum base (about 20%), a 30:1 (sugar/resveratrol) mix (about 62%), corn syrup (about 15%) and flavouring (about 1%).

12. A method of claim 11 wherein the ingredients are mixed together at a temperature of between 75° C. to 80° C.

13. A method of claim 12 wherein the ingredients are mixed together at a temperature of about 80° C.

14. A method of claim 1 whereby the subject's NAD level increases by between 30% and 50% within about 60 min of administration of the pharmaceutical composition.

15. A method of claim 14 whereby the subject's NAD level increases by between 35% and 45% within about 60 min of administration of the pharmaceutical composition.

16. A method of claim 15 whereby the subject's NAD level increases by 41% within about 60 min of administration of the pharmaceutical composition.

17. A method of claim 1 whereby the subject's raised NAD level remains between 30% and 40% above the baseline NAD level for between 1 and 4 hours and decreases at a rate of 1 ng/mL/hr to 3 ng/mL/hr.

18. A method of claim 17 whereby the subject's raised NAD level remains 35% above the baseline NAD level for 4 hours and decreases at a rate of 2 ng/mL/hr.

19. A method for the treatment of a disease or condition associated with either reduced NAD+ synthesis and/or increased NAD catabolism (usage) or increased requirement for NAD+ comprising:
administering to a subject a pharmaceutical composition comprising a therapeutically effective amount of resveratrol or a functionally equivalent analogue or derivative thereof; and
retaining the pharmaceutical composition in the subject's mouth so that absorption of the resveratrol occurs through the subject's buccal/sublingual membranes, thereby by-passing first pass metabolism by the liver.

20. A pharmaceutical composition when used to induce NAD+ synthesis in a subject, the composition comprising:
a therapeutically effective amount of resveratrol or a functionally equivalent analogue or derivative thereof;
whereby absorption of the resveratrol occurs through the subject's buccal/sublingual membranes, thereby by-passing first pass metabolism by the liver.

21. A pharmaceutical composition for administering a therapeutically effective amount of resveratrol or a functionally equivalent analogue or derivative thereof to a subject characterised in that absorption of the resveratrol occurs through the subject's buccal/sublingual membranes, thereby by-passing first pass metabolism by the liver; wherein the subject's NAD level increases by between 30% and 50% within about 60 min of administration of the composition.

22. A pharmaceutical composition for administering a therapeutically effective amount of resveratrol or a functionally equivalent analogue or derivative thereof to a subject characterised in that absorption of the resveratrol occurs through the subject's buccal/sublingual membranes, thereby by-passing first pass metabolism by the liver; wherein the subject's raised NAD level remains between 30% and 40% above the baseline NAD level for between 1 and 4 hours and decreases at a rate of 1 ng/mL/hr to 3 ng/mL/hr.
